(19) United States
(12) Plant Patent Application Publication
(10) Pub. No.: US 2019/0364714 P1
(43) Pub. Date: Nov. 28, 2019

(54) PHALAENOPSIS ORCHID PLANT NAMED 'PHALGELDYM'

(50) ;

(71) Applicant: ANTHURA B.V., BLEISWIJK (NL)

(72) Inventor: MARTINUS NICOLAAS GERARDUS VAN SWIETEN, UTRECHT (NL)

(21) Appl. No.: 15/932,941

(22) Filed: May 24, 2018

(51) Int. Cl. A01H 6/62

(52) U.S. Cl. PLT/311

(57) ABSTRACT

A new and distinct variety of Phalaenopsis plant named ‘PHALGELDYM’, particularly characterized by having small white flowers with white to light-yellow lips, 1 to 4 peduncles that are medium long, leaves that are oblong, and is propagated by meristem tissue culture, is disclosed.

Genus and species: Phalaenopsis hybrid.

Variety denomination: ‘PHALGELDYM’.

### BACKGROUND OF THE NEW PLANT

The present invention relates to a new and distinct cultivar of Phalaenopsis plant, botanically known as Phalaenopsis hybrid of the Orchidaceae family, commonly referred to as moth orchid, and hereinafter referred to by the variety name ‘PHALGELDYM’.

The new Phalaenopsis plant is a product of a planned breeding program conducted by the inventor in Bleiswijk, The Netherlands. The objective of this breeding program was to create a new Phalaenopsis plant with numerous attractive, small white flowers with white to light-yellow lips, suitable for potted plant production.

The new Phalaenopsis plant ‘PHALGELDYM’ is a result of cross-pollination made by the inventor in September 2008 in Bleiswijk, The Netherlands of the proprietary female, or seed parent, Phalaenopsis hybrid ‘01-8917’ (unpatented) with the proprietary male, or pollen parent, Phalaenopsis hybrid ‘01-8918’ (unpatented).

The new Phalaenopsis was selected by the inventor as a single plant within the progeny of the stated cross-pollination in a controlled greenhouse in Bleiswijk, The Netherlands in September 2011. Asexual reproduction of the new Phalaenopsis plant by meristem tissue culture since 2013 in Bleiswijk, The Netherlands, has demonstrated that the new variety reproduces true to type with all of the characteristics, as herein described, firmly fixed and retained through successive generations.

Plant Breeder's Rights for this variety have been applied for in Europe on Apr. 24, 2017. ‘PHALGELDYM’ has not been made publicly available or sold anywhere in the world more than one year prior to the effective filing date of this application.

### SUMMARY OF THE INVENTION

The following are the most outstanding and distinguishing characteristics of this new cultivar when grown under normal horticultural practices in Bleiswijk, The Netherlands and can be used to distinguish ‘PHALGELDYM’ as a new and distinct variety of Phalaenopsis plant.
1) Small white flowers with white to light-yellow lips;
2) 1 to 4 peduncles;
3) Peduncle is medium long; and
4) Shape of the leaf is oblong.

### DESCRIPTION OF THE PHOTOGRAPHS

This new Phalaenopsis plant is illustrated by the accompanying photographs which show the overall plant habit including blooms, buds and foliage of the plant; the colors shown are as true as can be reasonably obtained by conventional photographic procedures. The photographs were taken in a greenhouse in Bleiswijk, The Netherlands, from 50-week old plants in April 2018. Colors in the photographs may differ from the color values cited in the detailed botanical description, which accurately describe the actual colors of the new variety.

FIG. 1 shows the overall plant habit, including blooms, buds and foliage of ‘PHALGELDYM’.

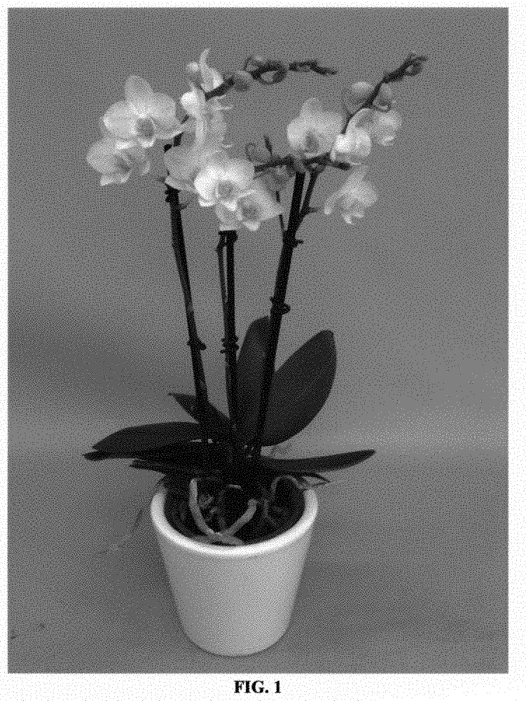

FIG. 2 shows a close-up of a flower of ‘PHALGELDYM’.

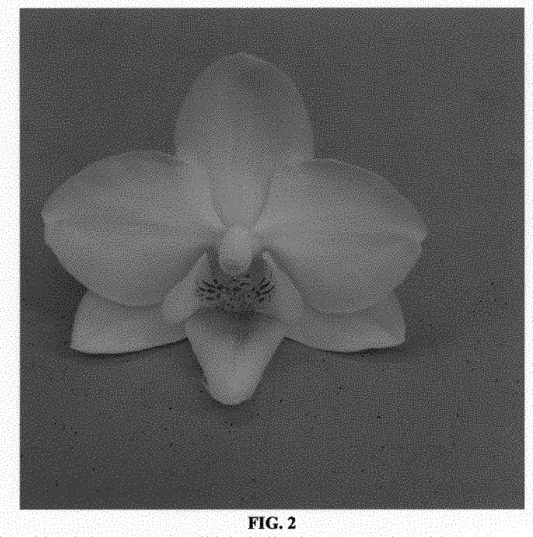

FIG. 3 shows an overhead view of the leaves of ‘PHALGELDYM’.

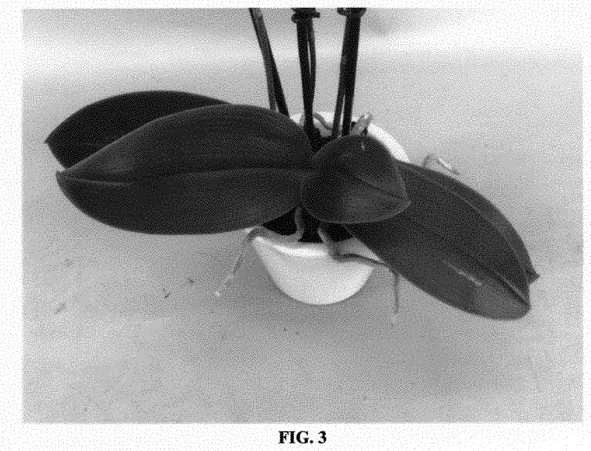

### DESCRIPTION OF THE NEW VARIETY

The following detailed description sets forth the distinctive characteristics of ‘PHALGELDYM’. Plants of the new Phalaenopsis have not been observed under all possible environmental conditions. The phenotype may vary somewhat with variations in environment such as temperature, light intensity and day length, without, however, any variance in genotype. The chart used in the identification of colors described herein is the R.H.S. Colour Chart of The Royal Horticultural Society, London, England, 2015 edition, except where general color terms of ordinary significance are used. The color values were determined under 4000-6000 lux natural light in a greenhouse in Bleiswijk, The Netherlands. Observations and measurements were made in April 2018 on flowering plants which were planted in 9 centimeter (diameter) pots. After in-vitro propagation, the plants were grown in nursery trays for 18-20 weeks, followed by transplantation to 9 centimeter (diameter) pots and grown in a greenhouse between 27° C. to 29° C. for 25 weeks, continued by a cooling period of 6 weeks between 18° C. to 20° C. and 11 weeks in a greenhouse of 21° C. Flowering occurs after 42-weeks in a 9 centimeter pot.

### DETAILED BOTANICAL DESCRIPTION

Classification:
   Family.—Orchidaceae.
   Botanical.—Phalaenopsis hybrid.
   Common name.—Moth orchid.
   Variety name.—‘PHALGELDYM’.
Parentage:
   Female parent.—Phalaenopsis cultivar ‘01-8917’ (unpatented).
   Male parent.—Phalaenopsis cultivar ‘01-8918’ (unpatented).
Propagation:
   Type.—Meristem tissue culture.
Roots:
   Root description.—Greyed-green colored roots (RHS 190B/C) with branching lateral roots having light green (RHS 145B) colored root tips.
Plant:
   Commercial crop time to flowering.—Following asexual propagation (in-vitro), the rooted cuttings grow for 18-20 weeks. After transplantation into 9 cm pots, the plants are finished after 40 to 42 weeks.
   Growth habit of peduncle.—Standard, green leaves, raceme, sometimes panicle.
   Height (from soil level to top of inflorescence).—Approximately 28.0 cm to 33.0 cm.
   Width (measured from leaf tips).—About 22.0 cm to 24.0 cm.
   Vigor.—Moderate.
Leaves:
   Mature leaves.—Quantity per plant: 8 to 10 leaves are produced before flowering. Length (fully expanded): 11.0 cm to 13.0 cm. Width: 5.5 cm to 6.5 cm. Shape: Oblong. Base shape: Moderately elongated. Apex: Obtuse unequal. Leaf blade angle with the petiole (measured from the horizontal position): Between 15 degrees and 30 degrees. Leaf margin: Entire. Color: Upper surface: RHS 147A. Lower surface: RHS 147B and diluting dark red (RHS 187A). Texture: Smooth. Thickness: 1.8 mm to 2.3 mm. Venation: Pattern: Parallel. Color of the midvein: Upper surface: RHS 147A. Lower surface: RHS 146A.
Peduncle:
   Quantity per plant.—1 to 4.
   Number of flowers per peduncle.—10 to 16.
   Length.—28.0 cm to 33.0 cm.
   Diameter.—3.0 mm to 3.3 mm.
   Strength.—Moderate.
   Aspect.—Upright to slightly pendant.
   Texture.—Smooth.
   Color.—Green (RHS 144A) with a touch of light brown (RHS N199A).
   Internode length.—2.5 cm to 3.5 cm.
   Callosities.—None.
Inflorescence description:
   Appearance.—Upright to slightly pendant, raceme, sometimes panicle inflorescence with bilaterally symmetrical flowers that open in succession beginning with the lowermost flower.
   Inflorescence size.—Height (from base to tip): 120.0 mm to 150.0 mm.
   Flowering time.—First flowers can be expected 7 to 8 months after planting in a 9 cm (diameter) pot.
   Flower.—Height: 37.0 mm to 42.0 mm. Diameter: 40.0 mm to 45.0 mm. Depth of lip: 13.0 mm to 15.0 mm.
   Flower longevity.—On the plant: 7 to 11 weeks.
   Fragrance.—Absent.
   Flower bud.—Average size: Small. Length: 14.0 mm to 16.0 mm. Width: 12.0 mm to 14.0 mm. Shape: Egg shaped. Color: Light yellow-green (RHS 145C).
   Petals.—Arrangement: Open/free. Shape: Semi-circular. Apex: Emarginated asymmetric. Margin: Slightly undulated. Length (from base to tip): 19.0 mm to 21.0 mm. Width: 18.0 mm to 20.0 mm. Color (when fully opened): Upper surface: Basic color: White (RHS NN155C). Over color: Absent. Lower surface: Basic color: White (RHS NN155C). Over color: Absent.
   Dorsal sepal.—Shape: Elliptic. Apex: Obtuse symmetric. Margin: Entire. Length (from base to tip): 22.0 mm to 24.0 mm. Width: 15.0 mm to 17.0 mm. Color (when fully opened): Upper surface: Basic color: White (RHS NN155C). Over color: Absent. Lower surface: Basic color: White (RHS NN155C). Over color: Absent.
   Lateral sepals.—Shape: Ovate. Apex: Acute asymmetric. Margin: Entire. Length (from base to tip): 22.0 mm to 24.0 mm. Width: 14.0 mm to 16.0 mm. Color (when fully opened): Upper surface: Basic color: White (RHS NN155C). Over color: Very small dots (RHS 71A) at the base. Lower surface: Basic color: White (RHS NN155C). Over color: Absent.
   Labellum (lip).—Whiskers: Present. Length of whiskers: 4.0 mm to 6.0 mm. Color of whiskers: White (RHS NN155C). Pubescence on the lip: Absent.
   Lateral lobe.—Shape: Type V (as described in the International Union for the Protection of New Varieties of Plants (UPOV) Test Guidelines for Phalaenopsis); spatulate. Margin: Slightly undulated. Length: 13.0 mm to 15.0 mm. Width: 8.0 mm to 10.0 mm. Color: White (RHS NN155C); striped (RHS 174A) at the base.
   Apical lobe.—Shape: Triangular. Margin: Entire. Length: 13.0 mm to 15.0 mm. Width: 11.0 mm to 13.0 mm. Color: Light yellow (RHS 8C/D) with small orange-yellow (RHS 165C) spots at the base and white (RHS NN155C) toward the whiskers.
   Callus.—Average size: Small. Height: 3.0 mm to 4.0 mm. Length: 3.0 mm to 4.0 mm. Width: 2.0 mm to 3.0 mm. Color: Yellow (RHS 12A/B) dotted (RHS 174A).
Reproductive organs:
   Column.—Length: 6.0 mm to 8.0 mm. Diameter: 3.3 mm to 3.6 mm. Color: White (RHS NN155C).
   Pollinia.—Quantity: 2. Diameter: 0.6 mm to 0.8 mm. Color: Orange (RHS 24A).
   Ovary.—Length: 12.0 mm to 14.0 mm. Diameter: 1.6 mm to 1.8 mm.
   Pedicel.—Length: 29.0 mm to 31.0 mm. Diameter: 2.0 mm to 2.2 mm. Color: Light green (RHS 144A/B) at the base and white (RHS NN155C) toward the flower.
Disease, pest, and stress resistance: No specific resistance or susceptibility observed to pathogens and pests common to Phalaenopsis to date.
Fruit and seeds: Fruit and seed development has not been observed on plants of the new Phalaenopsis to date.

### COMPARISON WITH PARENTAL LINES AND MOST SIMILAR VARIETIES

The female parent plant of ‘PHALGELDYM’, cultivar ‘01-8917’, is no longer in existence, so a meaningful comparison cannot be made.

The male parent plant of ‘PHALGELDYM’, cultivar ‘01-8918’, is no longer in existence, so a meaningful comparison cannot be made.

‘PHALGELDYM’ is most similar to the commercial Phalaenopsis plants named ‘PHALGALDAN’ (U.S. Plant patent application Ser. No. 15/932,588) and ‘PHALGLODAL’ (U.S. Plant patent application Ser. No. 15/932,540). ‘PHALGELDYM’ differs from the commercial variety ‘PHALGALDAN’ in that ‘PHALGELDYM’ has apical lobes that are light yellow with small orange-yellow spots at the base and white toward the whiskers, no over color on the lateral sepal lower surfaces, and a horizontal whisker position, whereas ‘PHALGALDAN’ has white apical lobes, very light green lateral sepal lower surfaces with very light purple tips, and a vertical whisker position. Additionally, ‘PHALGELDYM’ has shorter leaves than ‘PHALGALDAN’.

‘PHALGELDYM’ differs from the commercial variety ‘PHALGLODAL’ in that ‘PHALGELDYM’ has no over color on the lateral sepal lower surfaces, whereas ‘PHALGLODAL’ has slightly light yellow-green lateral sepal lower surfaces with purple midvein toward the tip. Additionally, ‘PHALGELDYM’ has shorter leaves and shorter plants than ‘PHALGLODAL’.

I claim:

1. A new and distinct variety of Phalaenopsis plant named ‘PHALGELDYM’, substantially as described and illustrated herein.
